(19) United States
(12) Patent Application Publication
(10) Pub. No.: US 2005/0154030 A1
(43) Pub. Date: Jul. 14, 2005

(54) Antimicrobial composition

(75) Inventor: Stephen A. Payne, Charlotte, NC (US)

Correspondence Address: KENNEDY COVINGTON LOBDELL & HICKMAN, LLP, 214 N. TRYON STREET, HEARST TOWER, 47TH FLOOR, CHARLOTTE, NC, 28202, US

(73) Assignee: Microban Products Company, Huntersville, NC (US)

(21) Appl. No.: 11/009,148

(22) Filed: Dec. 10, 2004

Related U.S. Application Data
(60) Provisional application No. 60/529,164, filed on Dec. 12, 2003.
(60) Provisional application No. 60/551,426, filed on Mar. 9, 2004.
(60) Provisional application No. 60/568,821, filed on May 6, 2004.

(51) Int. Cl. A61K 31/44; A61K 31/4196; A61K 31/4178; A61K 31/14

(52) U.S. Cl. 514/358; 514/383; 514/397; 514/643

(57) ABSTRACT

An antimicrobial composition comprising a microemulsion for imparting antimicrobial characteristics to numerous products. The microemulsion can be aqueous or non-aqueous. A method of making the antimicrobial composition is also disclosed.

### CROSS-REFERENCES TO RELATED APPLICATIONS

This application is entitled to the benefit of, and claims priority to provisional U.S. Patent Application Ser. No. 60/529,164, filed on Dec. 12, 2003, provisional U.S. Patent Application Ser. No. 60/551,426, filed on Mar. 9, 2004, and provisional U.S. Patent Application Ser. No. 60/568,821, filed on May 6, 2004, each of which is incorporated herein by reference in its entirety.

### FIELD OF THE INVENTION

The invention relates to the field of antimicrobial protection. More specifically, the invention relates to a composition for imparting built-in and long lasting antimicrobial characteristics to products as diverse as paint, vinyl siding, treated wood, and paper for gypsum wallboard or insulation. In particular, the invention pertains to a novel and synergistic emulsion of a quaternary ammonium antimicrobial agent and at least two other non-metallic antimicrobial agents.

### BACKGROUND OF THE INVENTION

The field of providing products with built-in antimicrobial protection has grown tremendously over the past several years. What once started out as a premium or novel option for high-end consumer products and medical devices has now grown into a mainstream characteristic found in many consumer products. Consumers can go to any home improvement center and see dozens if not hundreds of products that claim some degree of resistance to microbiological growth or contamination. Some major retailers have specific sections devoted to such antimicrobial products.

One of the challenges faced in all built-in antimicrobial applications is matching an effective antimicrobial agent with a particular product. For example, one antimicrobial agent may work well in interior applications (e.g., interior paint) yet be unsuitable for some outdoor applications (e.g., house siding). Similarly, an agent that works well against one type of microbe (e.g., fungi) may not work against another type of microbe (e.g., bacteria).

Similarly, one may anticipate that a combination of a particular bactericide and fungicide will work to provide the degree of antimicrobial protection sought for a particular product then discover that the two active agents are incompatible or must be delivered to the product separately and thus more expensively.

Accordingly, imparting antimicrobial characteristics to particular products is not simply a matter of pulling an antimicrobial agent off of a shelf and adding it to an existing product. Many variables must be considered and sometimes a commercially acceptable solution (i.e., effective and economically acceptable) is not readily apparent. Furthermore, as the field of built-in antimicrobial protection grows, each new product presents researchers with a new set of problems.

Therefore, a continuing need exists for new antimicrobial compositions that can be added to the arsenal of weapons used to fight the proliferation of microbes on consumer and industrial products.

### SUMMARY OF THE INVENTION

The new and useful antimicrobial composition of the present invention can impart antimicrobial characteristics in a wide range of products.

In one embodiment, an antimicrobial composition comprises an aqueous microemulsion comprising a quaternary ammonium antimicrobial agent, a phenol, a first non-metallic antimicrobial agent, a second non-metallic antimicrobial agent, and water.

In another embodiment, an antimicrobial composition comprises a non-aqueous microemulsion comprising a quaternary ammonium antimicrobial agent, a first non-metallic antimicrobial agent, a second non-metallic antimicrobial agent, and a glycol.

Also provided is a method of manufacturing a new and useful antimicrobial compositions. In one embodiment, the method is for making an antimicrobial composition comprising an aqueous microemulsion. The method comprises blending a phenol with a quaternary ammonium antimicrobial agent, admixing a quantity of a first non-metallic antimicrobial agent, admixing a quantity of a second non-metallic antimicrobial agent, and admixing a quantity of water.

In another embodiment, the method is for making an antimicrobial composition comprising a non-aqueous microemulsion. This method comprises admixing a quantity of a first non-metallic antimicrobial agent with a glycol, admixing a quantity of a quaternary ammonium compound in the presence of heat, and admixing a quantity of a second non-metallic antimicrobial agent.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a photograph after inoculation with a fungal species of a wood sample having no treatment with an antimicrobial composition.

FIG. 2 is a photograph after inoculation with a fungal species of a wood sample treated with an antimicrobial composition in accordance with the present invention.

FIG. 3 is a photograph after inoculation with a fungal species of a wood sample treated with an antimicrobial composition in accordance with the present invention.

FIG. 4 is a photograph after inoculation with a fungal species of a wood sample treated with an antimicrobial composition in accordance with the present invention.

FIG. 5 is a photograph after inoculation with a fungal species of a wood sample treated with an antimicrobial composition in accordance with the present invention

### DESCRIPTION OF THE PREFERRED EMBODIMENTS

As used herein, the terms “microbe” or “microbial” should be interpreted to encompass any of the microscopic organisms commonly studied by microbiologists. Such organisms include, but are not limited to, bacteria and fungi as well as other single-celled organisms such as mold, mildew and algae. Viral particles and other infectious agents are also included in the term microbe.

The term “antimicrobial” includes biostatic activity, i.e., where the proliferation of microbiological species is reduced or eliminated, and true biocidal activity where microbiological species are killed. For ease of discussion, this detailed description may make reference to bacteria and antibacterial agents. This method of presentation should not be interpreted as limiting the scope of the invention in any way.

The term “efficacy,” as used herein, is defined as the characteristic of inhibiting the growth of a microbe on a substrate.

The term “non-metallic,” as used herein, means antimicrobial agents, other than quaternary ammonium compounds, that do not contain or utilize metal ions (e.g., Ag, Cu).

In broad terms, the invention is an antimicrobial composition comprising a microemulsion of a quaternary ammonium antimicrobial agent and two different types of non-metallic antimicrobial agents. The microemulsion can be in two forms. One is an aqueous microemulsion well suited for aqueous systems. The other is a non-aqueous microemulsion well suited for non-aqueous or organic solvent type systems.

Turning now to more specific embodiments of the invention, one embodiment of the invention is an aqueous antimicrobial composition that can impart antimicrobial characteristics to many different products. In its most basic form, this embodiment of the invention comprises an aqueous microemulsion. The microemulsion comprises a quaternary ammonium antimicrobial agent, a phenol, a first non-metallic antimicrobial agent, a second non-metallic antimicrobial agent, and water. Interestingly, the composition need not contain the volatile alcohols (e.g., ethanol) that are usually used to form emulsions of quaternary ammonium antimicrobial agents. Each of these elements, and other preferred and optional elements, will be discussed in more detail below.

Quaternary ammonium antimicrobial agents include, but are not limited to, N-alkyldimethyl benzyl ammonium saccharinate, 1,3,5-Triazine-1,3,5(2H,4H,6H)-triethanol; 1-Decanaminium, N-decyl-N, N-dimethyl-, chloride (or) Didecyl dimethyl ammonium chloride; 2-(2-(p-(Diisobuyl)cresosxy)ethoxy)ehyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl) benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61 % C11, 23% C14); alkyl dimethyl benzyl ammonium chloride (61% C12, 23% C14); alkyl dimethyl benzyl. ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethy benzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis (2-hydroxyethyl) octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethyylbenzyl ammonium chloride; and n-octadecyl dimethyl benzyl ammonium chloride.

In particularly preferred embodiments the quaternary. ammonium antimicrobial agent comprises a dimethylbenzyl ammonium compound such as N-alkyl dimethylbenzyl ammonium saccharinate. N-alkyl dimethylbenzyl ammonium saccharinate is commercially available from Stepan Chemical Company of Northfield, Ill., under the tradename ONYXIDE™ 3300. This particular form of ONYXIDE™ is approximately 95% active and is a solid at room temperature but will form a liquid at elevated temperature. It is light yellow-orange in color and is insoluble in water.

The phenol may comprise an alkyl phenol having at least one an alkyl group selected from the group consisting of C₇ alkyls, C₈ alkyls, C₉ alkyls, C₁₀ alkyls, and C₁₁ alkyls.

In most preferred embodiments the alkyl phenol comprises an alkyl phenol having a C₉ alkyl group.

Alkyl phenols suitable for use in the invention are available commercially from a number of sources. A particularly preferred commercially available alkyl phenol is sold by Dow Chemical Company under the tradename TRITON™ X-207.

Alternatively, the phenol could comprise a styrenated phenol. Two examples of styrenated phenols acceptable for use with the invention are CHROMASIST WEZ and STANDAPOL HS. Both are available from Cognis Corporation of Cincinnati, Ohio. Both are identified by CAS #3217120. Technical data sheets indicate that the primary difference between the two is the level of ethoxylation.

The different phenols that may be employed in the practice of the invention is an example of both the flexibility of the invention and the choices that one skilled in the art may have to make in the practice of the invention. Those skilled in the art will recognize that one phenol may work better in one application (e.g., paints) than another (e.g., kraft paper). Fine tuning of the invention to suit a particular process should not limit the scope of the invention in any way. Those skilled in the art will be able to make the small necessary adjustments to adapt the invention to their particular process without undue experimentation.

The non-metallic antimicrobial agents used in the practice of the invention are preferably selected from the azole family of antimicrobial agents. Generally speaking, azoles comprise a large class of compounds characterized by a five-membered ring which contains an atom of nitrogen and at least one other non-carbon atom (e.g., nitrogen, oxygen, and sulphur). Certain azoles exhibit antimicrobial (i.e., antifungal) properties. Triazoles are a subclass of azoles that are often used as antimicrobial agents.

Two of the more well known antimicrobial triazoles are propiconazole and tebuconazole. The chemical name for propiconazole (CAS No. 50207-90-1) is 1[[2-(2,4-dichlorophenyl)-4-propyl-1,3-dioxolan-2-yl]methyl]-1H-1,2,4-triazole. The chemical name for tebuconazole (CAS No. 107534-96-3) is α-[2-(4-chlorophenyl)ethyl]-α-(1,1-dimethylethyl)-1H-1,2,4-triazole-1-ethanol.

Tebuconazole is commercially available from a number of sources. A particularly preferred form of tebuconazole is available from Bayer Corporation under the tradename PREVENTOL™ A8. Similarly, propiconazole is available from a number of sources. A particularly preferred form of propiconazole is available from Janssen Pharmaceutica under the tradename WOCOSEN™ TECHNICAL.

In particularly preferred embodiments, the first and second non-metallic antimicrobial agents are propiconazole and tebuconazole. Propiconazole will be referred to as the first non-metallic agent and tebuconazole will be referred to as the second non-metallic agent.

A small quantity of a glycol may be present in the composition according to the invention if desired. Glycols can add to the stability of the emulsion and provide other benefits such as retarding foam. Preferred glycols include alkyl glycols with polyethylene glycol, polypropylene glycol, and dipropylene glycol being preferred.

Water makes up the other primary component of the claimed antimicrobial composition.

The antimicrobial composition according to the invention may comprise other additives. Two such additives are anti-foaming agents and anti-freezing agents.

Some phenols used in the practice of the invention can be susceptible to foaming depending upon the particular application. Therefore, it is envisioned that many commercial embodiments of the invention will contain anti-foaming agents.

The composition according to the invention may be used to treat or preserve wood such as timber, framing, lumber, and pressure treated wood. It is particularly desirable to preserve the wood and to protect against mold or fungus although it is also desirable to protect against bacteria. The composition according to the invention may be used to surface treat wood such as timber, framing, lumber, and pressure treated wood.

For example, the composition according to the invention may be used to treat kraft paper. Such paper is a component of several construction materials including but not limited to gypsum wallboard and insulation.

Wallboard paper is quite thick and one method of treating this type of paper, but not the only method, is to add the antimicrobial composition to the water box, a device toward the end of the paper process that returns a certain quantity of water to the paper after the paper has undergone heat based drying.

The water box is often agitated due to the continuous movement of paper through it. This agitation can cause foaming upon the addition of the claimed antimicrobial composition. Generally, the presence of foam is detrimental to the water box portion of a paper process. Therefore, an anti-foaming agent is added to the composition according to the invention when the invention is used to treat paper at the water box.

Insulation paper is normally thinner than wallboard paper and can be treated using a spraybar. Foam buildup in the spraybar can be detrimental to a process so an antifoaming agent can be used there as well.

Preferred anti-foaming agents are ethoxylated co-polymers of polyethylene glycol. In some instances, the commercial formulations of phenols may contain a quantity of an antifoaming agent. For example, TRITON™ X-207 contains a small quantity of polyethylene glycol. Accordingly, if TRITON™ X-207 is the source of the phenol additional antifoaming agent may not be needed.

Likewise, anti-freezing agents may be added to the composition according to the invention. They are used to keep the composition from freezing or becoming too viscous during transport in cold weather. In most instances a glycol will serve the function of an anti-freezing agent. A preferred anti-freezing agent is dipropylene glycol.

One of the benefits of the present invention is that it provides a novel and improved platform for taking advantage of the antimicrobial properties of various azoles and tebuconazole and propiconazole in particular. For some years now those skilled in the art have been aware of a general synergy between tebuconazole and propiconazole. In other words, by using tebuconazole and propiconazole in combination, one can achieve a greater antimicrobial effect for a given amount of antimicrobial agent.

Tebuconazole and propiconazole are also well known for their hydrophobicity and difficulty of use in an aqueous environment. They resist forming aqueous emulsions and to the extent they can be forced into some type of aqueous emulsion the emulsions tend to be delicate and are easily destabilized (e.g., the actives form crystals in water and precipitate out or they form distinct phases with water).

The composition according to the invention, however, provides a stable aqueous microemulsion of azoles, particularly tebuconazole and propiconazole. This composition can be as dilute as about 0.02 wt. % of combined azole (e.g., 100 ppm of tebuconazole and 100 ppm of propiconazole) or as concentrated as about 50 wt. % combined azole. This characteristic provides great flexibility in how the composition may be used.

Although not to be bound by any particular theory, it is believed that the quaternary ammonium compound in conjunction with the phenol provides a stabilizing effect on the two azoles. In particular, dimethylbenzyl ammonium compounds such as ONYXIDE™ 3300 have been shown to provide a stabilizing effect on aqueous emulsions having combinations of azoles such as tebuconazole and propiconazole.

Turning now to more specific embodiments of the invention, the relative quantities of each of the listed components may vary to accommodate particular process requirements. The versatility of the invention is discussed in more detail below but generally it should be recognized that the optimal formulation for one product (e.g., a polymer) may be different from the optimal formulation for another product (e.g., wood treatment or paper). Accordingly, each of the listed components may be present in different amounts depending upon the particular needs of the user. Again, those of ordinary skill in the art are fully capable of making these adjustments without undue experimentation.

In preferred embodiments the quaternary ammonium antimicrobial agent is present in the overall composition in an amount between about 1 wt. % and 20 wt. %. In particularly preferred embodiments, the quatemary ammonium antimicrobial agent is present between about 2 wt. % and 16 wt. % of the overall composition, most preferably between about 3 wt. % and 9wt. %.

Likewise, the phenol (i.e., alkyl phenol, styrenated phenol, or combinations of them) is preferably present in the overall composition in an amount between about 30 wt. % and about 75 wt. % of the total composition. In preferred embodiments, the phenol is present in the composition between about 40 wt. % and about 70 wt. %, most preferably between about 45 wt. % and 65 wt. %.

The first non-metallic antimicrobial agent is preferably present in the overall composition in an amount between about 5 wt. % and about 45 wt. %, more preferably between about 15 wt. % and about 30 wt. %. As noted previously, the first non-metallic antimicrobial agent is preferably tebuconazole.

The second non-metallic antimicrobial agent is preferably present in the overall composition in an amount between about 5 wt. % and about 45 wt. %, more preferably between about 15 wt. % and about 30 wt. %. As noted previously, the second non-metallic antimicrobial agent is preferably propiconazole.

A small quantity of a glycol may be present in the composition according to the invention. Preferred glycols include alkyl glycols with polyethylene glycol, polypropylene glycol, and dipropylene glycol being preferred. Dipropylene glycol is particularly preferred. It is anticipated that in most applications the quantity of glycol will be between about 0 wt. % and about 4 wt. %. of the total composition.

As mentioned earlier, if foaming is an issue an anti-foaming agent can be added. The anti-foaming agents suitable for use with the invention include those compounds commonly used as anti-foaming agents. In preferred embodiments, an additional amount of a glycol can be added as an anti-foaming agent. The glycol used as an anti-foaming agent can be more of the same glycol discussed previously or a different glycol.

A preferred anti-foaming agent is an ethoxylated co-polymer such as polyethylene glycol which is commercially available from Cognis Corporation. It is anticipated that in most applications the quantity of anti-foaming agent needed for successful practice of the invention will range between about 0 wt. % to about 3 wt. %, more preferably between about 0.5 wt. % and 1.5 wt. %. The quantity of antifo aming agent can be adjusted upwards or downwards depending upon the particular application. Those skilled in the art can readily determine the appropriate quantity of antifoaming agent to use without undue experimentation.

The remainder of the composition according to the invention comprises water. In preferred embodiments the quantity of water present in the claimed composition will be between about 0 wt. % and about 15 wt. %, more preferably between about 3 wt. % and about 9 wt. %. Again, the exact quantity of water will depend upon the particular application and one of ordinary skill in the art is capable of making the necessary adjustments.

Further embodiments of the invention include those products that incorporate the antimicrobial composition. Indeed, one of the novel aspects of the invention is that it serves as a very versatile tool for incorporating antimicrobial agents into a variety of diverse products. For example, the antimicrobial composition according to the invention has been shown to be particularly effective at imparting antimicrobial characteristics to paper used in the production of wallboard and insulation.

The antimicrobial composition according to the invention is an excellent tool for providing antimicrobial protection to products as diverse as wood, paint, polymers, paper and leather, with latex exterior paints and extruded vinyl (e.g., vinyl siding, vinyl windows) being particularly suitable for use with the invention. It is also anticipated that the antimicrobial composition according to the invention can be added to other solids such as ceramics and cementitious binders to impart antimicrobial characteristics.

The invention also encompasses a method of making an antimicrobial composition. In broad terms, the method according to the invention comprises blending a phenol with a quaternary ammonium antimicrobial agent. Heat may be necessary during this blending step and particularly if the phenol is an alkyl phenol and the quaternary agent is an N-alkyl dimethylbenzyl ammonium compound. To this phenol/quaternary mixture is admixed a quantity of a first non-metallic antimicrobial agent then a quantity of a second non-metallic antimicrobial agent. The first and second non-metallic agents can also be mixed together prior to mixing with the phenol/quaternary mixture. Toward the end of the process a quantity of water is added with mixing. Heat may be added to any-step if needed.

In preferred embodiments that use an alkyl phenol, the alkyl phenol (e.g., TRTION™ X-207) is usually blended with the quaternary ammonium antimicrobial agent (e.g., ONYXIDE™ 3300) in the presence of heat. The heat is applied because in many instances the quaternary ammonium antimicrobial agent is a solid at room temperature. If the quaternary ammonium antimicrobial agent is a liquid or available as a flowable composition heat may still be used to aid in the mixing. Care should be taken not to heat the admixture of alkyl phenol and quaternary antimicrobial agent to a point where there is unacceptable volatilization of either. In preferred embodiments the temperature is kept below about 200° C.

If TRITON™ X-207 is the alkyl phenol and ONYXIDE™ 3300 is the antimicrobial agent, a mixing temperature of between about 65° C. and about 75° C. is recommended. At this temperature the ONYXIDE™ 3300 melts into the TRITON™ X-207 to form a liquid.

The steps of admixing a first non-metallic antimicrobial agent and admixing a second non-metallic antimicrobial agent may occur separately. Alternatively, the first and second non-metallic antimicrobial agents can be admixed together then added to the quaternary ammonium antimicrobial agent.

The admixing of the non-metallic antimicrobial agents can be accomplished in the presence of heat, if needed. For example, WOCOSEN™ TECHNICAL. (i.e., propiconazole) is normally a very viscous liquid at room temperature. PREVENTOL™ A8 (i.e., tebuconazole) is normally a solid white powder at room temperature. Heating both of them will aid in the admixture to the quaternary ammonium/alkyl phenol mixture. In a preferred embodiment, the tebuconazole and propiconazole are mixed together in the presence of form a less viscous solution that can then be added to the quaternary ammonium/alkyl phenol mixture. In this embodiment the tebuconazole and propiconazole mixture is heated to between about 65° C. and 75° C. with stirring.

Heating can continue during the admixture of the non-metallic antimicrobial agents and the quaternary ammonium antimicrobial agents if needed. Glycols and any anti-foaming agents or anti-freezing agents such as those discussed previously can be added at this point.

Once the quaternary ammonium antimicrobial agent, phenol, and non-metallic antimicrobial agents are mixed heat may be removed. As the admixture cools to room temperature water is added with stirring.

If styrenated phenols are used in place of the alkyl phenols the method according to the invention is very similar to that recited above. The primary difference in using a styrenated phenol is that less heat may be required during mixing. Of course, the use of heat will always be somewhat dependent upon the precise active ingredients chosen by the practitioner.

The relative amounts of quaternary ammonium antimicrobial agent, phenol, non-metallic antimicrobial agents, and water utilized in the practice of the method according to the invention are the same as those discussed in relation to the composition according to the invention. Likewise, the relative amounts of anti-foaming agents, anti-freezing agents, and additional antimicrobial agents, if any, are the same as those discussed in relation to the composition according to the invention.

An alternative embodiment of this embodiment of the composition is particularly well suited for use in non-aqueous systems. In this embodiment the invention comprises a non-aqueous microemulsion. The microemulsion comprises a quaternary ammonium antimicrobial agent, a first non-metallic antimicrobial agent, a second non-metallic antimicrobial agent, and a glycol. The preferred quaternary ammonium antimicrobial agent, first non-metallic antimicrobial agent, and second non-metallic antimicrobial agent are the same as in the previous embodiment. Almost any glycol can be used in the practice of the invention but dipropylene glycol is a preferred glycol.

The preferred quantities of each element in the composition according to the invention is as follows: first non-metallic antimicrobial agent—about 10 wt. % to about 33 wt. %; second non-metallic antimicrobial agent—about 10 wt. % to about 33 wt. %; quaternary ammonium antimicrobial agent—about 0.33 wt. % to about 17 wt. %; glycol—about 17 wt. % to about 80 wt. %.

This method is generally similar to the method of making the aqueous microemulsion. The various components are mixed together in the appropriate quantities in the presence of heat, if needed. For example, several samples of the composition according to the invention were made in the following manner.

The first non-metallic antimicrobial agent (e.g., WOCOSEN™ TECHNICAL) was blended with dipropylene glycol under heat (approximately 65° C.) in the desired relative amounts. To this mixture was added the quaternary ammonium compound (e.g., ONYXIDE™ 3300) while maintaining heat. The second non-metallic antimicrobial agent (e.g., PREVENTOL A8) with heat and mixing. The entire mixture was then stirred under heat until a clear microemulsion was formed.

The above non-aqueous embodiment of the invention is well suited for imparting antimicrobial characteristics to products produced in a non-aqueous environment. Examples of such products include treated wood and lumber, solvent based paints and wallboard paper.

### EXAMPLES

### Example 1

An antimicrobial composition in accordance with the present invention was prepared as follows and is referred to as Composition A. The batch size was 50.9 lbs (23.1 kg).

TABLE 1

| Component | Quantity |
|---|---|
| WOCOSEN ™ | 9.7 lb (4.4 kg) |
| TECHNICAL | |
| ONYXIDE ™ 3300 | 2.4 lb (1.1 kg) |
| PREVENTOL ™ A8 | 9.5 lb (4.3 kg) |
| CHROMASSIST ™ WEZ | 29.3 lb (13.3 kg) |

The components were mixed in the order listed at a temperature of 85° C. (185° F.) in a reactor with ONYXIDE™ 3300 being added prior to PREVENTOL™ A8. The appearance of the composition was clear amber in color, and the composition had a slight odor. The percentage of solids was 59 to 65 %. The percentage active WOCOSEN™ TECHNICAL was 17.2 to 19%. The percentage active PREVENTOL™ A8 was 17.2 to 19%. The percentage active ONYXIDE™ 3300 was 4.3 to 4.75%. A 10% solution of concentrate in water was prepared and the pH was measured. The pH range was 7.5 to 8.5. The viscosity was measured using a Brookfield viscometer and was 500 cps at 25° C.

### Example 2

An antimicrobial composition in accordance with the present invention was prepared and is referred to as Composition B. The components were mixed in the order listed and under the same conditions as in Example 1.

TABLE 2

| Component | Quantity (%) | Active (%) |
|---|---|---|
| WOCOSEN ™ | 18.95 | 18 |
| TECHNICAL | | |
| PREVENTOL ™ A8 | 18.56 | 18 |
| ONYXIDE ™ 3300 | 9.47 | 9 |
| TRITON ™ X-207 | 48.02 | — |
| Water | 5.00 | — |

### Example 3

An antimicrobial composition in accordance with the present invention was prepared as follows and is referred to as Composition C. The components were mixed in the order listed.

The batch size was 450 lb (204 kg).

TABLE 3

| Component | Quantity (lb) |
|---|---|
| WOCOSEN ™ | 111.8 lb (50.71 kg) |
| TECHNICAL | |
| TRITON ™ X-207 | 113.2 lb (51.35 kg) |
| MDS-42 | 130.5 lb (59.19 kg) |
| CHROMASIST ™ WEZ | 94.5 lb (42.9 kg) |

The container for WOCOSEN™ TECHNICAL was heated in a hot water bath to lower its viscosity for mixing. The components were added in the order listed. The mixture of WOCOSEN™ TECHNICAL and TRITON™ X-207 was heated to 85° C. and stirred until the mixture was clear, and then the MDS42 and CHROMASIST™ WEZ were added with stirring. The mixture was then cooled to room temperature.

The appearance of the composition was clear amber in color, and the composition had a slight odor. The percentage active WOCOSEN™ TECHNICAL was 23.6%. The viscosity was measured using a Brookfield viscometer and was 790 cps at 25° C. A 10% solution of concentrate in water was prepared and the pH was measured. The pH range was 5 to 6. The specific gravity was 1.08.

### Example 4

Microbiological tests were conducted for treatment on wood in accordance with AATCC Test Method 30 Part III. The sample size was 25 mm×50 mm rectangle. The growth medium was Sabouraud Dextrose Aga (SDA). The samples were incubated at 30° C. ±2° C. for 18 to 24 hours. The zone size represents the percentage of coverage on the sample by the fungus with 100% representing being fully covered. With respect to the repetition growth column in Table 4, 0 represents no growth, 1 represents microscopic growth (visible under microscope), and 2 represents microscopic growth (visible under naked eye).

TABLE 4

| Sample | | | Repetition | Zone | Repetition | Zone |
|---|---|---|---|---|---|---|
| No. | Sample Description | Organism | One Growth | size | Two Growth | size |
| 1 | Control - no treatment | Aspergillus | 2 | 100 | 2 | 100 |
| | | Niger 6275 | | | | |
| 2 | Wood sprayed with | Aspergillus | 2 | 60 | 2 | 25 |
| | 0.275 g Comp. A and | Niger 6275 | | | | |
| | 100 g water | | | | | |
| 3 | Wood sprayed with | Aspergillus | 2 | 50 | 2 | 60 |
| | 0.275 g Comp. A and | Niger 6275 | | | | |
| | 100 g water. Let dry 24 | | | | | |
| | hours and then rinse | | | | | |
| | with water. | | | | | |
| 4 | Wood dipped in 0.275 g | Aspergillus | 2 | 15 | 2 | 15 |
| | Comp. A and 100 g | Niger 6275 | | | | |
| | water for 2 min. | | | | | |
| 5 | Wood dipped in 0.275 g | Aspergillus | 2 | 30 | 2 | 30 |
| | Comp. A and 100 g | Niger 6275 | | | | |
| | water for 2 min. Let | | | | | |
| | dry for 24 hrs and then | | | | | |
| | rinsed | | | | | |

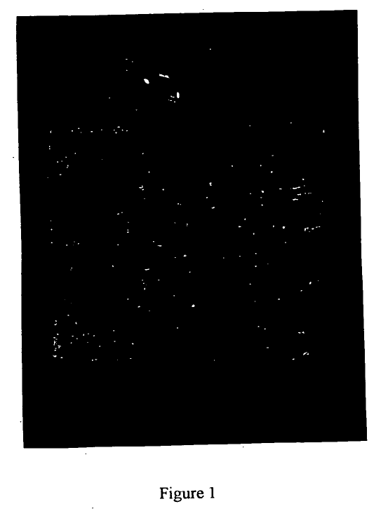

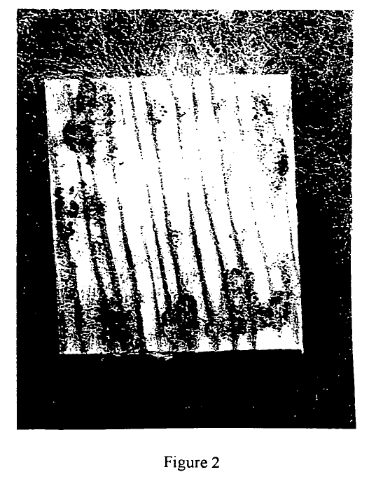

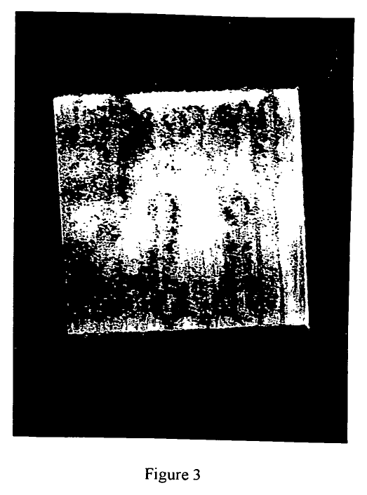

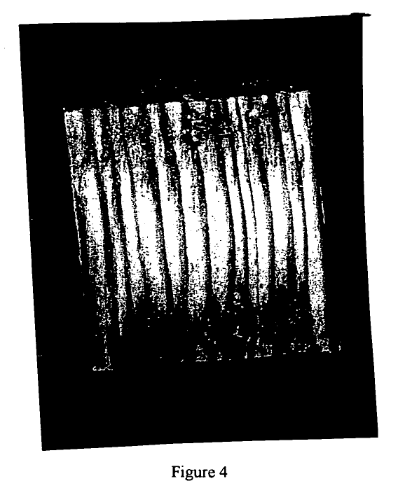

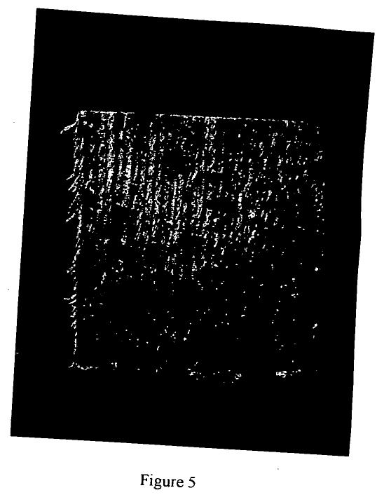

FIG. 1 is a photograph after inoculation with a fungal species of sample no. 1 having no treatment with an antimicrobial composition. FIG. 2 is a photograph after inoculation with a fungal species of sample no. 2 treated with an antimicrobial composition in accordance with the present invention. FIG. 3 is a photograph after inoculation with a fungal species of sample no. 3 treated with an antimicrobial composition in accordance with the present invention. FIG. 4,is a photograph after inoculation with a fungal species of sample no. 4 treated with an antimicrobial composition in accordance with the present invention. FIG. 5 is a photograph after inoculation with a fungal species of sample no. 5 treated with an antimicrobial composition in accordance with the present invention.

### Example 5

A test using standard test method D3273 for “Resistance to Growth of Mold on the Surface of Interior Coatings in an Environmental Chamber” was conducted. Wood samples were tested having a sample size of 1.5 inches×2.0 inches. The samples were rated for mold growth each week for four weeks on a 0 to 10 rating scale. Zero indicated 100% coverage which is total ftngal disfigurement and a 10 indicated complete absence of fungal disfigurement. The table set forth below represents the percentage of surface defaced by fungal growth. The samples were tested at varying percentages of a Composition D which is comprised of a 4:1 ratio of Composition C to Tebuconazole powder (100% concentration). Note that the asterisk represents that microscopic examination showed that defacement was not fungal.

TABLE 5

| | | | | | | | | | | Final ASTM | Final ASTM |
|---|---|---|---|---|---|---|---|---|---|---|---|
| | | Week 1 | Week 1 | Week 2 | Week 2 | Week 3 | Week 3 | Week 4 | Week 4 | 3273 Rating | 3273 Rating |
| Sample ID | Rep. | Front | Back | Front | Back | Front | Back | Front | Back | (Front) | (Back) |
| Control | 1 | 90 | 90 | 90 | 90 | 100 | 90 | 100 | 100 | 0 | 0 |
| Control | 2 | 90 | 90 | 90 | 90 | 95 | 90 | 100 | 100 | 0 | 0 |
| Control | 3 | 90 | 90 | 90 | 90 | 95 | 90 | 100 | 100 | 0 | 0 |
| 0.05% | 1 | 0 | 0 | 0 | 1 | 0 | 5 | 0 | 5 | 10 | 9 |
| Comp. D | | | | | | | | | | | |
| 0.05% | 2 | 0 | 1 | 0 | 1 | 0 | 1 | 5 | 5 | 9 | 9 |
| Comp. D | | | | | | | | | | | |
| 0.05% | 3 | 0 | 5 | 0 | 10 | 0 | 10 | 1 | 10 | 9 | 9 |
| Comp. D | | | | | | | | | | | |
| 0.1% | 1 | 0 | 5 | 1 | 10 | 1 | 10 | 0* | 1* | 10 | 9 |
| Comp. D | | | | | | | | | | | |
| 0.1% | 2 | 0 | 0 | 0 | 5 | 1 | 5 | 1 | 5 | 9 | 9 |
| Comp. D | | | | | | | | | | | |
| 0.1% | 3 | 0 | 5 | 0 | 10 | 0 | 10 | 0 | 5* | 10 | 9 |
| Comp. D | | | | | | | | | | | |
| 0.2% | 1 | 0 | 0 | 0 | 5 | 0 | 10 | 1 | 10 | 9 | 9 |
| Comp. D | | | | | | | | | | | |
| 0.2% | 2 | 1 | 5 | 1 | 5 | 1 | 5 | 5 | 5 | 9 | 9 |
| Comp. D | | | | | | | | | | | |
| 0.2% | 3 | 0 | 5 | 0 | 5 | 0 | 5 | 1 | 10 | 9 | 9 |
| Comp. D | | | | | | | | | | | |
| 0.3% | 1 | 1 | 5 | 1 | 5 | 1 | 5 | 0* | 5 | 10 | 9 |
| Comp. D | | | | | | | | | | | |
| 0.3% | 2 | 0 | 5 | 1 | 5 | 1 | 10 | 1 | 10 | 9 | 9 |
| Comp. D | | | | | | | | | | | |
| 0.3% | 3 | 5 | 0 | 5 | 0 | 5 | 1 | 5 | 1 | 9 | 9 |
| Comp. D | | | | | | | | | | | |

### Example 6

Other compositions were prepared in accordance with the present invention and are set forth below. The components of each composition were mixed in the order listed and under the same conditions as in Example 2.

Composition E

| Component | Quantity (%) | Active (%) |
|---|---|---|
| WOCOSEN ™ TECHNICAL | 18.95 | 18 |
| PREVENTOL ™ A8 | 18.56 | 18 |
| ONYXIDE ™ 3300 | 9.47 | 9 |
| TRITON ™ X-207 | 43.02 | — |
| Water | 5.00 | — |
| Dipropylene glycol (DPG) | 5.00 | — |

Composition F

| Component | Quantity (%) | Active (%) |
|---|---|---|
| WOCOSEN ™ TECHNICAL | 18.95% | 18 |
| PREVENTOL ™ A8 | 18.56% | 18 |
| ONYXIDE ™ 3300 | 9.47% | 9 |
| TRITON ™ X-207 | 38.02% | — |
| Water | 5.00% | — |
| DPG | 10.00% | — |

Composition G

| Component | Quantity (%) | Active (%) |
|---|---|---|
| WOCOSEN ™ TECHNICAL | 18.95 | 18 |
| PREVENTOL ™ A8 | 18.56 | 18 |
| ONYXIDE ™ 3300 | 9.47 | 9 |
| TRITON ™ X-207 | 48.02% | — |
| DPG | 5.00% | — |

Composition H

| Component | Quantity (%) | Active (%) |
|---|---|---|
| WOCOSEN ™ TECHNICAL | 18.95% | 18 |
| PREVENTOL ™ A8 | 18.56% | 18 |
| ONYXIDE ™ 3300 | 9.47% | 9 |
| TRITON ™ X-207 | 43.02% | — |
| DPG | 10.00% | — |

It will therefore be readily understood by those persons skilled in the art that the present invention is susceptible of broad utility and application. Many embodiments and adaptations of the present invention other than those herein described, as well as many variations, modifications and equivalent arrangements, will be apparent-from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

1. An antimicrobial composition comprising an aqueous microemulsion, said aqueous microemulsion comprising:
a quaternary ammonium antimicrobial agent,
a phenol,
a first non-metallic antimicrobial agent,
a second non-metallic antimicrobial agent, and
water.

2. The antimicrobial composition according to claim 1, wherein
said quaternary ammonium antimicrobial agent is between about 1 wt. % and about 20 wt. % of the total composition;
said phenol is between about 30 wt. % to about 75 wt. % of the total composition;
said first non-metallic antimicrobial agent is between about 5 wt. % and about 45 wt. % of the total composition; and
said second non-metallic antimicrobial agent is between about 5 wt. % and about 45 wt. % of the total composition.

3. The antimicrobial composition according to claim 2, wherein said quaternary ammonium antimicrobial agent comprises an alkyl ammonium compound.

4. The antimicrobial composition according to claim 3, wherein said quaternary ammonium compound is an N-alkyl dimethylbenzyl ammonium compound.

5. The antimicrobial composition according to claim 2, wherein at least one of said first and second non-metallic antimicrobial agents are selected from the group consisting of azoles.

6. The antimicrobial composition according to claim 5, wherein said first non-metallic antimicrobial agent comprises tebuconazole and said second non-metallic antimicrobial agent comprises propiconazole.

7. The antimicrobial composition according to claim 2, wherein said phenol comprises an alkyl phenol having an alkyl group selected from the group consisting of C₇ alkyls, C₈ alkyls, C₉ alkyls, C₁₀ alkyls, and C₁₁ alkyls.

8. The antimicrobial composition according to claim 7, wherein said alkyl phenol comprises an alkyl phenol having a C₉ alkyl group.

9. The antimicrobial composition according to claim 2, wherein said phenol comprises a styrenated phenol.

10. The antimicrobial composition according to claim 1, further comprising an antifreeze agent.

11. The antimicrobial composition according to claim 1, further comprising an antifoaming agent.

12. The antimicrobial composition according to claim 1, wherein the first non-metallic antimicrobial agent is propiconazole.

13. The antimicrobial composition according to claim 1, wherein the second non-metallic antimicrobial agent is tebuconazole.

14. A paint comprising the antimicrobial composition of claim 1.

15. A paper comprising the antimicrobial composition of claim 1.

16. An antimicrobial composition comprising a non-aqueous microemulsion, said non-aqueous microemulsion comprising:
a quaternary ammonium antimicrobial agent,
a first non-metallic antimicrobial agent,
a second non-metallic antimicrobial agent, and
a glycol.

17. The antimicrobial composition according to claim 16, wherein
said quaternary ammonium antimicrobial agent is between about 0.33 wt. % and about 17 % by weight of the total composition,
said first non-metallic antimicrobial agent is between about 10 wt. % and about 33 wt. % by weight of the total composition,
said second non-metallic antimicrobial agent is between about 10 wt. % and about 33 wt. % by weight of the total composition, and
said glycol is between about 17 wt. % and about 80 wt. % of the total composition.

18. The antimicrobial composition according to claim 17, wherein said quaternary ammonium antimicrobial agent comprises an alkyl ammonium compound.

19. The antimicrobial composition according to claim 18, wherein said alkyl ammonium compound is an N-alkyl dimethylbenzyl ammonium compound.

20. The antimicrobial composition according to claim 17, wherein the non-metallic antimicrobial agents are selected from the group consisting of azoles.

21. The antimicrobial composition according to claim 20, wherein the first non-metallic antimicrobial agent comprises propiconazole and the second non-metallic antimicrobial agent comprises tebuconazole.

22. A method of making an antimicrobial composition comprising an aqueous microemulsion, the method comprising:
blending a phenol with a quaternary ammonium antimicrobial agent,
admixing a quantity of a first non-metallic antimicrobial agent,
admixing a quantity of a second non-metallic antimicrobial agent, and
admixing a quantity of water.

23. The method according to claim 22, wherein the quaternary ammonium antimicrobial agent comprises an alkyl ammonium compound.

24. The method according to claim 23, wherein said alkyl ammonium compound comprises an N-alkyl dimethylbenzyl compound.

25. The method according to claim 22, wherein
the quantity of blended quaternary ammonium antimicrobial agent is between about 1 wt. % and about 20 wt. % of the total composition;
the quantity of blended phenol is between about 30 wt. % and about 70 wt. % of the total composition;
the quantity of admixed first non-metallic antimicrobial agent is between about 5 wt. % and about 45 wt. % of the total composition; and
the quantity of admixed second non-metallic antimicrobial agent is between about 5 wt. % and about 45 wt. % of the total composition.

26. The method according to claim 22, further comprising admixing an antifoaming agent.

27. The method according to claim 22, further comprising admixing an antifreeze agent to the composition.

28. The method according to claim 22, wherein the phenol comprises an alkyl phenol having an alkyl group selected from the group consisting of C₇ alkyls, C₈ alkyls, C₉ alkyls, C₁₀ alkyls, and C₁₁ alkyls.

29. The method according to claim 22, wherein at least one of the non-metallic antimicrobial agents is an azole.

30. The method according to claim 29, wherein the first non-metallic antimicrobial agent is propiconazole and the second non-metallic antimicrobial agent is tebuconazole.

31. The method according to claim 22, wherein the phenol is a styrenated phenol.

32. The method according to claim 28, wherein blending the quaternary ammonium antimicrobial agent with the alkyl phenol occurs under the application of heat.

33. The method according to claim 32, wherein admixing the first and second non-metallic antimicrobial agents occurs under the application of heat.

34. The method according to claim 33, wherein admixing water occurs in the absence of added heat.

35. The method according to claim 31, wherein blending the quaternary ammonium antimicrobial agent with the styrenated phenol occurs under the application of heat.

36. The method according to claim 22, further comprising applying the antimicrobial composition to a substrate.

37. The method according to claim 36, wherein the substrate is selected from the group consisting of wood, polymers, fabrics, ceramics, paper, paints, and leather.

38. The method of making an antimicrobial composition comprising a non-aqueous. microemulsion, the method comprising:
admixing a quantity of a first non-metallic antimicrobial agent with a glycol,
admixing a quantity of a quaternary ammonium compound under heat, and
admixing a quantity of a second non-metallic antimicrobial agent.

39. The method according to claim 38, wherein
the first non-metallic antimicrobial agent is an azole and the quantity of the first agent is between about 10 wt. % and about 33 wt. % of the total composition,
the second non-metallic antimicrobial agent is an azole and the quantity of the second agent is between about 10 wt. % and about 33 wt. % of the total composition,
the glycol is present between about 17 wt. % and about 80 wt. % of the total composition, and
the quaternary ammonium compound is present between about 0.33 wt. % and about 17 wt. % of the total composition.

40. The method according to claim 39, wherein the glycol is dipropylene glycol and the quaternary ammonium compound comprises an N-alkyl dimethylbenzyl compound.

41. A wallboard having antimicrobial properties, said wallboard comprising:
paper,
a quaternary ammonium antimicrobial agent,
a phenol,
a first non-metallic antimicrobial agent,
a second non-metallic antimicrobial agent, and
a glycol.

42. Insulation having antimicrobial properties, said insulation comprising:
insulating material,
paper,
a quaternary ammonium antimicrobial agent,
a phenol,
a first non-metallic antimicrobial agent, and
a second non-metallic antimicrobial agent.

43. Treated wood having antimicrobial properties, said treated wood comprising:
a quaternary ammonium antimicrobial agent,
a first non-metallic antimicrobial agent, and
a second non-metallic antimicrobial agent.

44. The treated wood according to claim 43, wherein the first non-metallic antimicrobial agent is propiconazole.

45. The treated wood according to claim 43, wherein the second non-metallic antimicrobial agent is tebuconazole.

46. The treated wood according to claim 43, further comprising a phenol.

47. The treated wood according to claim 43, further comprising a glycol.

48. The treated wood according to claim 43, further comprising water.
